(19) United States
(12) Patent Application Publication
(10) Pub. No.: US 2003/0008338 A1
(43) Pub. Date: Jan. 9, 2003

(54) Assay methods and kits therefor

(75) Inventors: Sharon Patricia Mary Crouch, Nottingham (GB); Kevin John Slater, Nottingham (GB)

Correspondence Address: BANNER & WITCOFF, 1001 G STREET N W, SUITE 1100, WASHINGTON, DC, 20001, US

(73) Assignee: LumiTech (UK) Limited, Nottingham (GB)

(21) Appl. No.: 10/005,189

(22) Filed: Dec. 7, 2001

(30) Foreign Application Priority Data
Dec. 15, 2000 (GB) 0030726.4

(51) Int. Cl. C12Q 1/66; C12Q 1/48

(52) U.S. Cl. 435/8; 435/15

(57) ABSTRACT

The invention relates to methods for detecting the cytotoxic activity of an effector, such as a cytotoxic T lymphocyte, on a non-microbial target cell. Detection of cytotoxic activity is preferably achieved by detecting adenylate kinase release using photometric methods.

### BACKGROUND

1. Field of the Invention

The invention relates to assay methods and kits for performing assays. In particular, the assays are useful for determining cytolysis and cytotoxicity.

2. Description of Related Art

The most widely used technique to evaluate cell-mediated cytotoxicity is a radioactive assay based on prelabelling of target cells with chromium-51. Chromium is rapidly incorporated into live cells as the chromate ion. It is reduced in the cytoplasm to the chromic ion which, when released from dead cells, cannot be reincorporated. Although this is a very sensitive method, the use of chromium has the major disadvantage that it involves the handling of radioactive elements. Working with ⁵¹Cr as a radioisotope that emits gamma rays requires separate facilities, ⁵¹Cr being hazardous to human health. Connected to this is the environmental load that the radioactive waste material brings with it. Moreover, there are also technical problems: in some target cells and especially in fresh non-transformed cells, ⁵¹Cr uptake is often low and spontaneous release high. There remains a need for efficient labelling of normal proliferating cells.

Over the years, several attempts have been made to replace the radioactive ⁵¹Cr release assay with a non-radioactive method, but these attempts have been largely unsuccessful.

Bachy et al., J. Imm. Methods 230 (1999) 37-46 report the use of β-galactosidase release as an alternative to radioactive chromium release in cytotoxic T-cell assays. According to page 38 of Bachy et al., other proposed methods which rely on detection of the release of naturally expressed intracellular enzymes, e.g. alkaline phosphatase, are not adapted to the evaluation of effector cell (e.g. cytotoxic T lymphocyte, CTL) function because these endogenous enzymes are also expressed in the effector cells, leading to high backgrounds. To avoid these problems Bachy et al reasoned that it was better to assay an enzyme which was released exclusively by target cell synthesis, namely β-galactosidase.

Beta galactosidase is an enzyme from the bacterium Escherichia coli, which enzyme can be assayed using a luminescent substrate. The Bachy et al assay involves the transient expression of β-galactosidase obtained by infection of target mammalian cells with recombinant β-gal vaccinia virus on the day before use in the cytotoxicity assay. Incubation of target cells with effector cells (CTLs) on the day following infection resulted in release of β-galactosidase which was dependent on the infection time (typically 4 hours) and the effector/target cell ratio.

Clearly, the need to produce transient β-gal expression in target cells by infection with recombinant β-gal vaccinia vectors is a serious drawback of the Bachy et al method.

Nociari et al., J. Imm. Methods 213 (1998) 157-167 also report a fluorometric assay to evaluate cell-mediated cytotoxicity, which is an alternative to assays based on radioactive chromium release. The assay utilises a fluorometric compound known as “alamarBlue”, which compound is a metabolic indicator of viable cells and becomes fluorescent upon mitochondrial reduction. Specific lysis of target mammalian cells by effector cells (CTLs) is quantified by comparing the total number of viable cells in wells containing effector and target cells, together with wells where target and effector cells are seeded separately. It is stated on page 165 that, although cell-mediated cytotoxicity can be detected after six hours using the alamarBlue assay, 24 hours is the optimal incubation period. The requirement for such a prolonged incubation step is a drawback of the alamarBlue assay.

A loss of cell integrity, through damage to the plasma membrane, results in the leakage of a number of factors from cells cultured in vitro into the surrounding medium. A number of cytoplasmic proteins are present in the cytoplasm of the cell at essentially constant concentrations. Upon damage by cytotoxic agents and/or cells, a target cell will release these proteins into the culture supernatant. We have recognised that adenylate kinase (AK) is a ubiquitous protein present in all eukaryotic cells, and that the amount of enzyme within a cell remains relatively constant. Furthermore, we have shown that measurement of this enzyme allows the accurate and sensitive determination of cytotoxicity and cytolysis.

AK measurements have been used for low level detection of microbial contamination in a sample. WO 94/17202 describes a method in which the amount of AK is estimated by its ability to convert adenosine diphosphate (ADP) to adenosine triphosphate (ATP) and relating that to the presence or amount of micro-organism. ATP is detected using the luciferin/luciferase bioluminescent reagent system to provide a photometrically detectable signal indicative of the amount of ATP in the sample. It is mentioned that, as with any amplified assay, the sensitivity of the AK assay is limited by the purity of the reagents. ADP at a purity of higher than 99.95% is required. Moreover, AK is generally present as a contaminant in luciferase preparations and its presence is likely to significantly affect the interpretation of the results since the aim is to measure very low AK levels in samples. Hence, highly purified reagents are required for these methods.

### BRIEF DESCRIPTION OF THE DRAWINGS

FIG. 1: The effect of increasing concentrations of ADP on light output (measured as relative light units, or RLUs) immediately after addition of AMR. The AK activity was from heat treated K562 cells at 10⁴/ml.

FIG. 2: The effect of increasing magnesium acetate concentrations on light output immediately after addition of AMR (Time 0) and after 5 minutes. The background levels of AK activity in complete medium, ie no contributions from cells is shown by the lines. The results represent the means of triplicate wells.

FIG. 3 The effect of cell number on AK activity. The cells at room temperature acted as the controls for the heat treated cells. The results represent the means of triplicate experiments±standard deviation (SD).

FIG. 4: The effect of different sample volumes on determination of AK activity. The assays with cells or supernatants were performed in triplicate wells of a 96 well white walled microtitre plate, and the data represents the means±SD.

FIG. 5: The effect of increasing necrotic cell number in the presence of healthy cells. The results shown are the means of triplicate wells±SD.

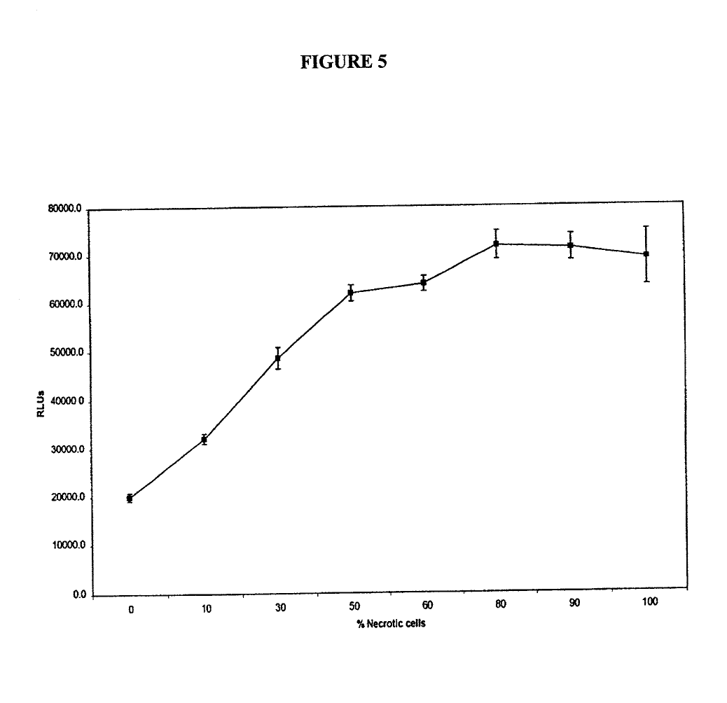

FIG. 6: The effect of 1000 nM camptothecin on AK release in U937 cells. The results represent the means of triplicate wells±SD.

FIG. 7: The effect of increasing concentrations of ionomycin on K562 cells. The results represent the means of triplicate wells±SD.

There was a significant increase in AK activity with all concentrations of the agent.

### DETAILED DESCRIPTION OF THE INVENTION

The present invention seeks to provide methods, kits and reagents, which can be used to measure cytotoxic activity in which the above problems are avoided or substantially mitigated.

Assay methods and kits and reagents therefor according to the present invention are defined in the accompanying claims.

Definitions

“Cytotoxic activity”

This term is intended to include any damage to a cell whereby AK is released from the cell. Such damage will cause the membrane of the cell to lose its integrity.

“Cellular effector”

This term includes any cell which is capable of cytotoxic activity against a target cell. For example, the cellular effector may be a cytotoxic T lymphocyte (CTL), natural killer cell, tumour infiltrating lymphocytes and/or lymphokine activated killer cell and/or cytokine induced killer cell. These cells can either be activated in vitro, for example the activation of NK cells by interleukin-2, or the cells can be isolated from the spleen of animals such as transgenic mice. Cells which are compromised in terms of their cytotoxicity can also be isolated from virally infected patients, and used in experiments to show the compromised function of these cells. The most commonly used cells are isolated from human, mice and rat, although bovine cells and non-mammalian cells may also be used.

“non-cellular effector”

This term is intended to embrace any agent which is not a cellular effector, but which is capable of cytotoxic activity against a target cell. Such agents include physical agents such as heat treatment, freeze/thaw and sonication; and chemical agents such as dexamethasone, camptothecin, ionomycin etc. There are a vast number of agents that are capable of inducing cytolysis. These include a large number of chemotherapeutic agents, and also include naturally occurring toxins and venoms. Bacterial toxins can also be used in cytotoxicity experiments. The following is a list of commonly used chemotherapeutic agents: doxorubicin, carmustine, bleomycin sulphate, daunorubicin, actinomycin-D, cytosine arabinoside, cyclophosphamide, dacarbazine, fluarabine, fluorouracil, idarubicin, ifosphamide, methotrexate, mithramycin, mitoquasone dihydrochloride, vinorelbine, trimetrexate glucuronate, pentostatin, mitoxantrone, vincristine sulphate, taxol, carboplatin, cisplatin, vinblastine, etoposide, streptozotocin.

Also within the definition of non-cellular effector are cytokines produced naturally as a result of infection, injury and/or tissue damage, together with complement components that can induce complement-mediated and complement-dependent cytotoxicity. An example of such naturally produced compounds is tumour necrosis factor (TNF).

“non-microbial cell”

This term is intended to embrace cells derived from any non-microbial source, for example from mammals, fish, crustaceans, plants etc. Preferably the non-microbial cell is an animal cell, for example derived from a mammal, especially a human, bovine, or murine source, or a cell line therefrom. Most of the cell lines used as targets in cell mediated cytotoxicity (K562, Daudi, Raji, U937) that are available can be supplied either by the ECACC or the ATCC. Target cells can be isolated from solid tumours, either from animal models or from human patients, examples of which include ovarian carcinoma, prostate carcinoma, colon adenocarcinoma, melanoma, bladder tumours and small cell lung cancers (all human). Mouse models include prostate carcinoma, mastocytoma, kidney sarcoma, lymphoma, pancreatic β cells (diabetic studies), H-15 melanoma cells, and various other mouse tumour models. Also within the above definition are rat hepatoma cells, cells infected in vitro with viruses (e.g. bovine leukaemia virus infected fibroblasts), or parasites (e.g. canine 030-D cells infected with leishmania parasite) and also cells infected with intracellular parasites.

With respect to the cytotoxicity assays with physical and chemical agents, skilled persons will appreciate that almost any non-microbial cell can be used provided they can be kept in culture long enough to perform the experiments. Suitable cells can also be bought commercially from companies such as Clonetics in the USA.

Cancer cell lines may also be obtained from the National Cancer Institute of the NIH, Biological Research Laboratory, Bethesda, Md. 20892, USA (see Boyd et al, Principles & Practices of Oncology, 3(10): October 1989 and Curt, Oncologist, 1(3): II-III, 1996).

“multi-well microtitre plate”

This term is intended to embrace apparatus, which comprises a plurality of reaction vessels or wells linked together in the form of a plate. Each well has a small volume, usually 250 to 300 μl in a 96 well plate, 60 to 70 μl in a 384 well plate and 6-8 μl in a 1536 well plate. At present, the most common plates have 96 wells, but plates having 384 and 1536 wells are known and useful according to the invention.

“High throughput screening”

This term covers the screening of large numbers of chemically generated and naturally-derived products for generating leads to pharmaceutical products. Compounds are put into groups for screening using microtitre plate technologies.

“Substantially constant”

This term is used in relation to emitted light intensity. It is intended to include the meaning that the light intensity does not vary significantly over the same time period as is taken to carry out the light intensity measurements. As a non-limiting example, the term is intended to include the meaning that the rate of change of emitted light intensity is less than 5% per minute, and preferably less than 3% per minute.

The bioluminescent reagent can be any of the luciferin/luciferase general type. The active substrate is D-luciferin or a derivative thereof. U.S. Pat. No. 5,374,534 discloses D-luciferin derivatives, which may be used with luciferase in the method of the invention.

Any other derivative can be used. The luciferase enzyme is preferably obtained naturally, especially from fireflies and most especially Photinus pyralis. However, the source of the luciferase is not critical, so long as it reacts with luciferin (or a derivative thereof) and ATP in the bioluminescent reaction. Examples are luciferases from Luciola cruciata, Diptera spp. and Coleoptera spp.

Synthetic, for example, recombinant luciferase can be used in the invention. It is described by Devine et al., Biochemica et Biphysica Acta 1173, No. 2, 121-132 (1993) and in European Patent 301,541 and U.S. Pat. No. 5,583,024.

In addition, mutant luciferases may be used which have been mutated so as to confer favourable properties that enhance the performance of the enzyme, including thermostability and resistance to low pH and high salt solutions. Suitable mutant luciferases can be obtained from Kikkoman Biochemicals, Japan. Other exemplary mutant luciferases suitable for use in the methods of the invention are disclosed in White et al Biochem J. 319, 343-350 (1996), Squirrel et al J. Defence Science 2, 292-297 (1997), Karp and Oker-Blom Biomolecular Engineering 16,101-104 (1999), Branchini et al Biochemistry 38, 13223-13230 (1999), Branchini et al Biochemistry 39, 5433-5440 (2000), Tatsumi et al Anal. Biochem. 243, 176-180 (1996), WO 98/46729, WO 96/22376, WO 99/02697, EPO 449 621 B, U.S. Pat. Nos. 5,330,906, 6,074,859, and WO 95/18853.

Skilled persons will recognise that there are a number of applications where determination of released AK according to the present invention may be used, these include:

Detection of soluble mediator induced cell death

Determination of cytolysis mediated by a physical inducer

Determination of the biocompatibility of a compound

Use in high throughput screening in drug discovery

Determination of cell mediated cytotoxicity

Determination of complement dependent cytotoxicity

Unlike the known use of AK for determination of low levels of bacteria, where there is a need for highly purified reagents, measurement of AK in cell cultures according to the present invention can be carried out using less specific requirements, due to the large amounts of AK that are being detected over and above background.

The assays of the invention are also non-destructive, in that detergents are not required to release the enzyme from the cell. If required, supernatants can be collected from the cultures and assayed for AK activity, allowing for further tests to be completed on the cultured cells. However, the assays of the invention can also be performed in the presence of cellular material, as this does not interfere significantly with the end point detection system. Thus, assays of the invention may be used in conjunction with one or more other assays, for example as part of a multi-test screen of a sample of non-microbial cells, to determine other properties of the cells. Exemplary assays that may be used include the ApoGlow3 adenylate nucleotide ratio assay for detection of apoptosis, necrosis and cell proliferation (LumiTech, Nottingham, UK).

For example, having removed a small sample (usually 10-20 μl) of conditioned media, it is possible to perform other methods of cell viability including ATP determination on the cells after lysis. If required, it is also possible to use a number of tetrazolium salts such as MTT and XTT. Further methods that may easily be carried out on the cells remaining in the wells include reporter gene assays. Measurement of AK gives a good indication of the effect of various assay treatments on the viability of the cells.

The assays of the invention are suitable for use in 96 well microtitre plates, but will perform equally well in volumes consistent with 384 well plates. If desired, the assays can also be performed in larger volumes using cuvettes, or scaled down to volumes suitable for 1536 well plates.

It may also be possible to perform the assay on a solid surface, for example by determining activity of the enzyme harvested onto an appropriate membrane/filter surface. The assay endpoint is based on luminescence detection whereby AK has the ability to convert added ADP to ATP, the latter can then be detected using the luciferin/luciferase reaction which produces an amount of light proportional to the relative enzyme activity. The light can then be detected using an appropriate piece of equipment such as a luminometer, or beta counter (in out of co-incidence mode), or alternatively, a close coupled device (CCD) camera.

AK activity can be determined by addition of a single reagent to the cell culture supernatant/cell suspension, and monitoring the light output immediately after addition of the reagent, and if required, to monitor the increase in light out put over time until the reaction has plateaued. The time taken for the reaction to plateau varies according to the amount of AK that has leaked, or been released from the cells.

The preferred AK reagent contains all the components required to allow for successful conversion of ADP to ATP in the presence of the enzyme, plus the detection reagent in the form of an ATP monitoring reagent (hereinafter referred to as “AMR”). The constituents of AMR and the final reaction concentrations can be found in appendix 1.

Previously, it has been reported that there is a requirement for the addition of a source of magnesium ions to allow the reaction to proceed. We have shown that, other than the amount of magnesium present in the AMR and from the cells/media, it is not essential to add any excess magnesium to our detection reagent. In fact the addition of magnesium acetate above 10 mM results in a reduction in detectable light output.

It has also been suggested that high concentrations of highly pure ADP are required to drive the reaction towards ATP production. With both cells and a source of AK from rabbit muscle (myokinase) we have shown the amount of light to correlate in a linear fashion with the amount of ADP present, and with the high levels of light detectable from the induction of cytolysis, it can be preferable to use much lower concentrations (range 2.5 to 500 mM). At concentrations higher than 0.5 mM the reaction may proceed too quickly for accurate detection and comparisons between different conditions.

We found that the assays of the invention performed equally well whether the reagents were added separately, or as with the final reagent formulation, as an all-in-one step. The all-in-one addition is far more desirable for high throughput screening applications. All the initial work up experiments were performed using the reagents added separately to investigate the impact of different magnesium acetate and ADP concentrations. The results were then compared with the all-in-one reagent hereinafter referred to as (“AKR”).

The invention is advantageous because it permits cytolysis detection in cell culture populations in volumes that are convenient for microtitre plate technology. The reagent formulations used can be put together in a kit (or kits) format to allow for a simple one step method for the determination of AK activity. If desired the reagents can be added separately, and the kits supplied in this format. The invention provides a method for detecting AK that has been released from cells when the cell has lost plasma membrane integrity.

The reagents have been developed for use at high levels of AK in volumes (mentioned above) associated with non-microbial cell culture in microtitre plates. For example, the reagents may be used at AK levels associated with the culture of animal cells at seeding densities of between 100 to 100,000 cells per well.

Examples embodying aspects of the invention will now be described with reference to the accompanying figures:

### EXAMPLES

Materials and Methods

To determine the parameters for the assay, different cell types and different cell numbers were exposed to heat treatment. This caused disruption of the cell membrane and release of AK into the culture media. All experiments were performed with cells cultured or resuspended in complete media comprising RPMI-1640 medium (Labtech International, UK), supplemented with 10% v/v heat inactivated foetal calf serum (Labtech International, UK), 2 mM L-glutamine (Sigma, UK), 100 U/ml penicillin and 100 μg/ml streptomycin (Sigma, UK). To heat treat the cells, either K562 cells (available from ECACC No: 89121407) at 10⁴/ml, or Cem-7 cells (Keele University) at 10⁶/ml were suspended in 5 mls of complete medium, in sterile universals (Fahrenheit) and placed in a water bath for at least 30 minutes at 56° C. Increasing concentrations (diluted in double distilled H₂O) of essentially ATP free ADP (Biotrace, UK) in 50 μl volumes were added to triplicate wells of a 96-well, white opaque microtitre plate (Perkin Elmer Life Sciences). The concentration range was 2.5 to 333 μM, leading to final concentrations in the wells of 0.625 to 83.25 μM. Then 50 μl of 15 mM magnesium acetate (final concentration 3.75 mM) was added to each well. Then 50 μl of either heat-treated cell suspension or 501 U/ml myokinase (Calbiochem, UK) were added to each set of triplicate wells. The addition of the enzyme initiated the reaction and 50 μl of AMR was then added to each well and the light output determined immediately. The amount of light was determined over repeated 1-second integrals using a Labsystems Luminoskan, the reaction was followed every 90 seconds for 6 minutes. Although it has been reported that the reaction should be allowed to proceed to completion (after 5 minutes) before determining the light output, the large amounts of AK present with heat treated cells was causing the reaction to proceed in a linear fashion with concentrations of ADP above 5 μM (final concentration of 1.25 μM), even after 6 minutes. There was no difference in the sensitivity of the assay whether the results were read immediately after addition of all reagents or after 6 minutes.

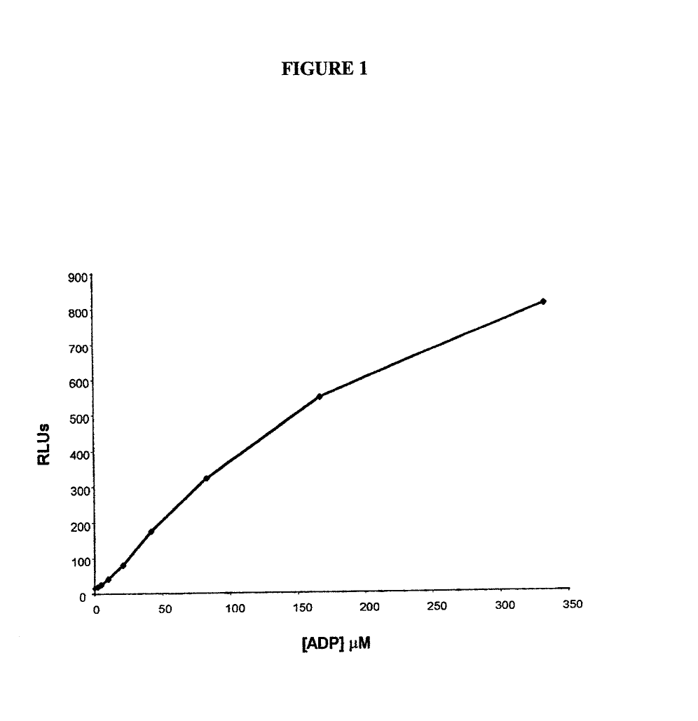

The results showed that there was a linear increase in light output proportionate to the amount of ADP present (See FIG. 1). However, it was the amount of AK that became the rate-limiting step due to the cell number present. The results obtained with the myokinase showed lower RLUs but there was still detectable activity above background with 2.5 μM added ADP. These experiments were repeated with Cem-7 cells at 106/ml, in this case the linearity of the curve continued after 167 μM ADP. With both cell types, experiments were performed where all the AK was released from the cells by the use of a Triton X100 based detergent (see appendix 1 for the formulation). These data confirmed that all the AK in these cell types at 10⁶/ml could be detected, even at the lowest concentrations of ADP.

Experiments were also performed where the detergent-induced lysate, containing the AK, was heated in the water bath at 56° C. This confirmed that the AK released was heat stable as there was no significant loss of activity.

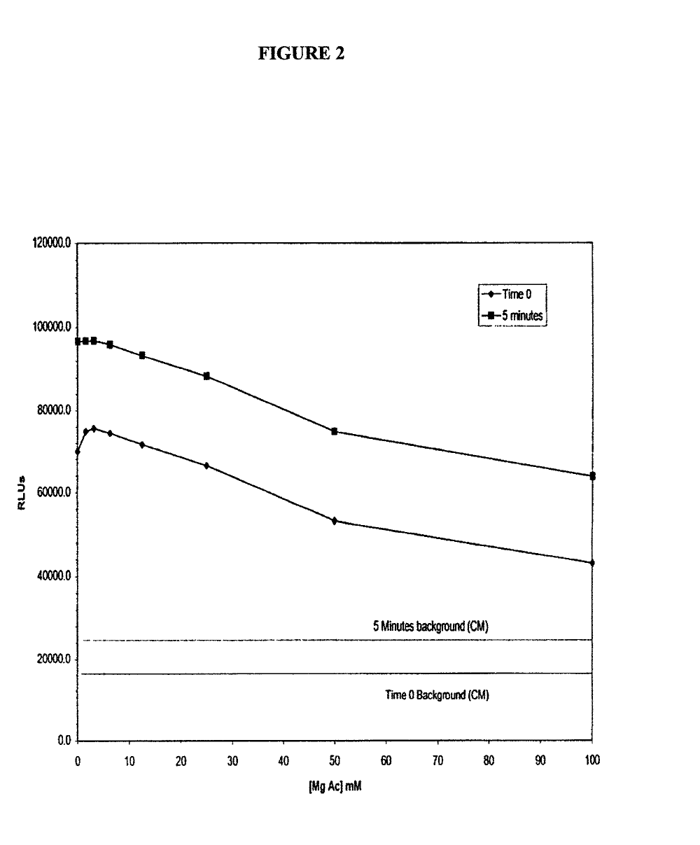

The AK released from heat-treated cells was used to test the requirement for additional magnesium ions, in the form of magnesium acetate. In these experiments, Cem-7 cells at 10⁶/ml were heated at 56° C. in complete medium in a water bath for at least 30 minutes. Triplicate wells of a white, opaque 96-well microtitre plate (Perkin Elmer Life Sciences) were set up with 50 μl of increasing concentrations of magnesium acetate, from 7.8 to 100 mM (final concentrations 1.95 to 25 mM plus 5 mM in the AMR). Control wells were also set up which had no additional magnesium acetate other than the 5 mM contributed from the AMR. To each well 50 μl of 0.3 mM ADP was added, followed by 50 μl of the Cem-7 heat-treated suspension and then immediately 501 μl of AMR. The plate was then read using a Wallac Microbeta Jet, with measurements taken at time 0 and after 5 minutes. Again, the RLUs were determined from 1 second integral readings at each time point. FIG. 2 shows an inhibitory effect of higher concentrations of magnesium acetate, but also demonstrates that there was no requirement for additional magnesium.

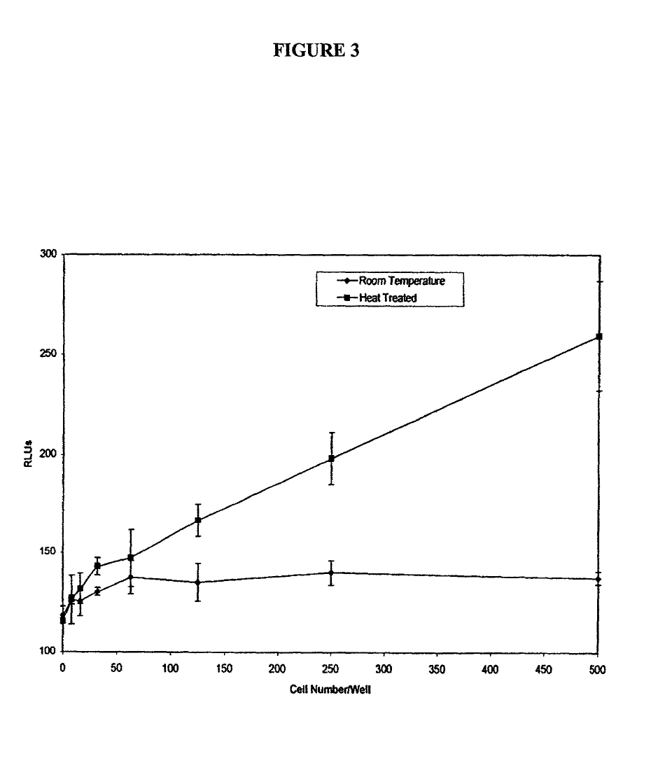

To show that the amount of AK was proportional to the lysed cells in culture, cells at serial doubling dilutions were heated at 56° C. for at least 30 minutes, and then AK activity determined in the cell suspension. For these experiments K562 cells were used at a starting cell number of 500 cells/well and diluted down to approximately 8 cells/well. The cells were added in 50 μl volumes, then 150 μl of an AKR containing AMR (relative constituents shown in appendix 1), Mg acetate at 2 mM (0.5 mM final plus the 5 mM from the AMR) and 0.3 mM ADP pH 7.0. Immediately after addition of these reagents (using an 8 channel pipette) the RLUs over 1-second integral were determined using the Labsystems Luminoskan luminometer, and read every 90 seconds for 6 minutes. The results showed a concentration dependent increase in light output with an increase in cell number. The results of the heat-treated cells were compared to a separate aliquot of cells that had been kept at room temperature, in this case the plasma membrane should have remained intact. FIG. 3 shows that AK activity did relate to cell damage. AK activity could be seen in the damaged cells both at time 0 and at 6 minutes after induction of the AK reaction.

The volume of cell suspension with AKR was also tested and revealed a volume dependent increase in AK activity. This also showed that the assay could be performed with very low sample volumes. In this case K562 cells at 10⁶/ml were heat treated as described previously, and then different volumes added to the white walled luminometer plates. The AKR at 150 μl was added via the injectors of the Wallac Microbeta Jet, and RLUs were determined every 2 minutes for up to 12 minutes. The results showed no significant differences between the different volumes with respect to the sensitivity of the assay. When the measurements were taken immediately after addition of the AKR by the luminometer injectors there was a correlation between sample volume and the amount of RLUs detected in the 1 second integral measurement. By 10 minutes the differences were less apparent, due to the kinetics of the ADP conversion to ATP, suggesting that measurement immediately after addition of the reagent was a more accurate determination of AK activity at high cell numbers.

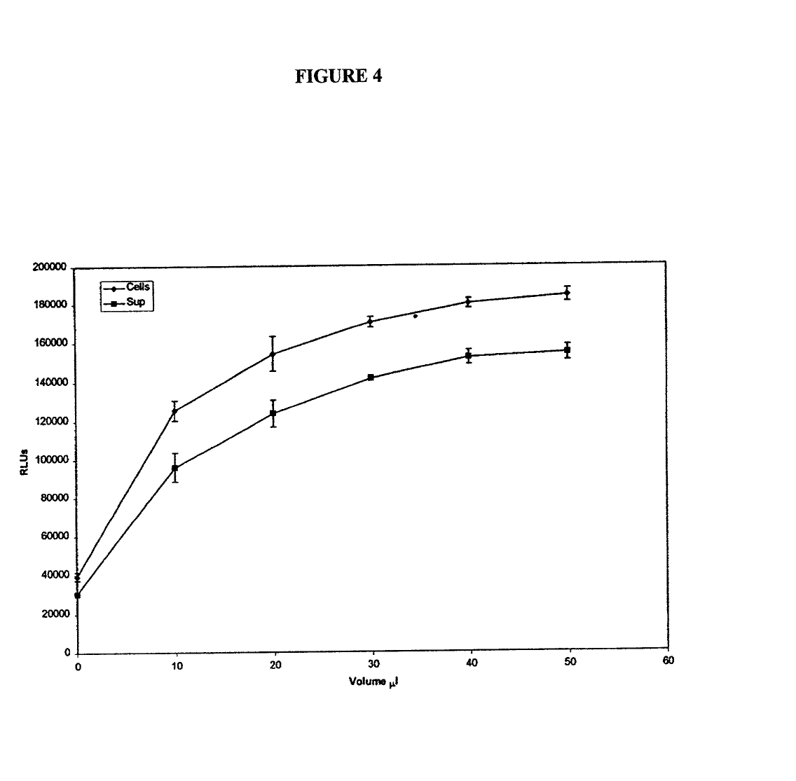

The experiments with different volumes were performed both with cellular material present and also on supernatants harvested from cell cultures, with the cells centrifuged out at ×400 g for 10 minutes. The resulting data demonstrated that the assay performed equally well whether the cells were present or not. These experiments also demonstrate that methods of the invention constitute a non-destructive method for determination of cytolysis, and having removed a sample from the culture, other tests can be performed on the same cell population if desired. FIG. 4 shows the results obtained for the different sample volumes. While the RLUs were different in the presence or absence of cellular material (Sup.) the sensitivity remained the same, with both curves running parallel to each other.

It was also possible to demonstrate that with 50 μl of cell supernatant, there was an increase in light output that corresponded with the volume of AKR used. These experiments showed a sensitivity down to 10 μl, and again demonstrated the applicability of this assay for 384 well plates. This meant that AK activity in K562 cells undergoing primary necrosis, induced by heating, could be detected with ADP levels as low as 5 nmoles and up to 16.65 nmoles. Even the presence of ADP at 5 nmoles increased the RLUs from 21±3 to 6486±996 (means of triplicates±SD). The greatest sensitivity was seen when light output over a 1 second integral was determined immediately after addition of the AKR.

All the data presented show high background activity, this is not associated with any background ATP levels, as any ATP in the media or other reagents represents less than 0.1% of the total light output. There was a certain amount of AK activity present in the foetal calf serum used in the complete medium, but this did not contribute significantly at the time=0 measurements, and only became evident 10 minutes after initiation of the reaction by addition of the AKR. Again this highlights the preference for taking a reading immediately after addition of AKR when working with cell cultures, where the amount of AK activity is much higher than that which has previously been investigated for microbiological investigations. The source of AK is from the luciferase reagent, although it is desirable to have AK-free luciferase, our data show that this is not essential. Also, due to the very small contribution of RLUs from ATP, there is no absolute requirement for ATP-free ADP. We have therefore optimized the assay system and reagents for use with the amount of AK that can be released from cytolytic cells in the range of 25 to 50,000 cells per well.

Most commonly, assay methods of the invention are performed in either 100 or 200 μl volumes in 96-well microtitre plates. In order to make the performance of the assay as simple as possible, it is preferable to culture cells in 100 μl volumes in a white-walled, clear bottomed tissue culture treated microtitre plate (Perkin Elmer Isoplates™). After induction of cytolysis the plate can be simply placed into a luminometer with injectors (such as the Wallac Microbeta Jet), and 150 μl of AKR dispensed automatically into each well and a 1-second integral reading taken immediately after addition of reagent.

Cem-7 cells at 10⁶/ml were heat treated to induce primary necrosis, and then cells were plated out in triplicate in 100 μl volumes with different mixtures of necrotic and healthy cells (i.e. those left at room temperature). These experiments were performed to confirm the ease of use with 100 μl, but also to show that the presence of viable cells did not interfere with the AK detection. AK activity was determined using the Wallac Microbeta Jet after injection of 150 μl of AKR. RLUs were read immediately after injection of reagent and at 2 minute intervals until 10 minutes. The results showed that there was no significantly detectable increase in AK activity with 100% necrosis vs 80% necrosis (the latter being in the presence of 20% viable). The 0% necrosis control represents a 100% viable population at the same cell number. The data show that it is possible to detect very low levels of cytolysis in the presence of a predominantly healthy population of cells.

Determination of Non-cellular, Soluble Agent-Induced Cytotoxicity

A number of different cell types have been tested for induction of cytotoxicity with soluble agents. Cytolysis is determined after incubation with the agent, when the cell membrane has lost integrity and AK can ‘leak’ out into the surrounding culture medium. The assay is performed by incubating non-microbial (e.g. mammalian) cells, either cell lines or primary cell types including isolated tumour cells, with the agonist(s) of choice. After an appropriate incubation time the cells are simply removed from the incubator and AK activity determined in the culture supernatant. If the cells have been cultured in clear bottomed, white walled plates (Perkin Elmer Life Sciences) in volumes no greater than 100 μl, then the plates are simply placed in a luminometer equipped with injection facilities and 150 μl of AKR added to each of the wells, where an integral reading of light output is measured over 1 second. When using luminometers without injectors the AKR can be added manually preferably using a multichannel pipette. The light output is read as soon after addition of reagent as possible. The increase in light output over the complete medium control is the result of the leakage of AK from damaged cells as a result of incubation with cytolytic inducing substances.

Example 1: We have investigated 37 different cell lines and models of apoptosis/necrosis induction. One of the first models investigated for this assay was the incubation of Cem-7 cells with dexamethasone. Two different experiments were performed: one experiment where cells at different cell numbers/well were incubated with one concentration of dexamethasone (500 nM, Sigma) for 48 and 72 hours, and another experiment where cells at 5000/well were incubated with increasing concentrations of the agent (0, 10, 25, 50, 75, 100, 150, 200 nM) for 72 hours. The cell suspensions were incubated in 100 μl in triplicate wells of a 96 well clear-bottomed white walled microtitre plate (Perkin Elmer Isoplate™). At the end of the incubation period the plates were assayed for AK activity using the Wallac Microbeta Jet, with 150 μl of AKR added and the RLUs (1 second integral) read at time 0 and every 5 minutes for 45 minutes. The results showed that at 48 hours there was no loss of membrane integrity and little AK activity above controls in the culture medium. However, at 72 hours the cells had undergone necrosis secondary to apoptosis, and there was a cell number dependent increase in AK activity. At 2500 cells per well the activity had increased by 71% from 15 913 RLUs at 48 hours to 27 266 RLUs at 72 hours.

The dexamethasone concentration curve revealed an increase in AK release from 11 772 RLUs in the controls to 16 936 with 200 nM dexamethasone. Even at a very low concentration of dexamethasone (25 nM), the detected AK activity was markedly greater than the control values.

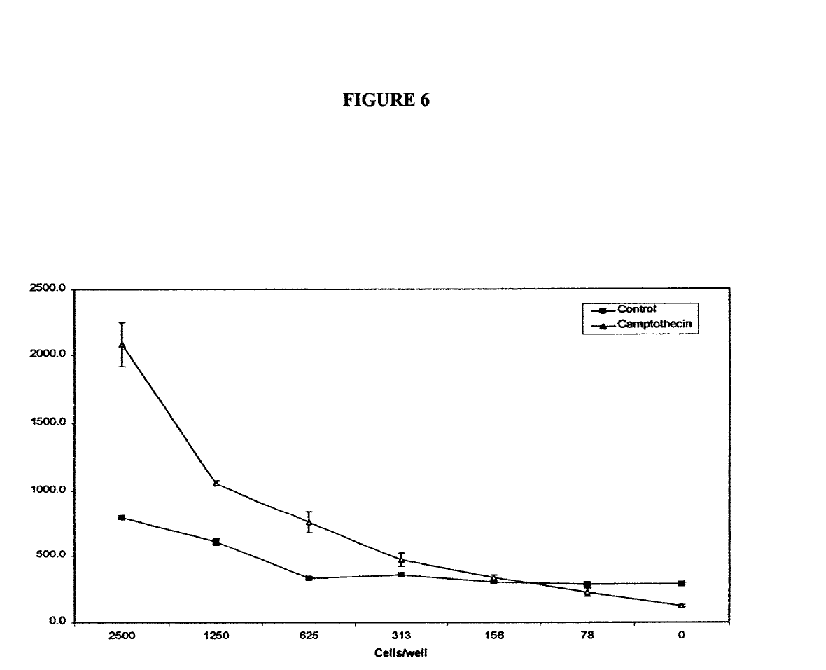

Example 2: A second model known to induce secondary necrosis is the incubation of U937 cells (ECACC No: 87010802) with camptothecin (Sigma). The cells were set up in triplicate wells of a white Isoplate™ (Perkin Elmer Life Sciences) at serial doubling dilutions of cells with 1000 nM camptothecin. The cells were then incubated with the drug for 4 hours and AK activity was determined as outlined in example 1. The data showed that the increase in activity after 4 hours was seen with cell numbers above 156/well (see FIG. 6). In the control cells there was a slight increase in AK activity over the complete medium control, but the increase in the presence of the drug was much greater, nearly 3 fold (2.6).

The experiment using increasing concentrations of camptothecin (from 50 to 1000 μM) with the cells at 2500/well also showed a concentration dependent increase in enzyme activity with cytolysis appearing at 100 μM. The light output was monitored at time 0, after 5 and 10 minutes, with the clearest concentration dependent effect seen with the time 0 reading.

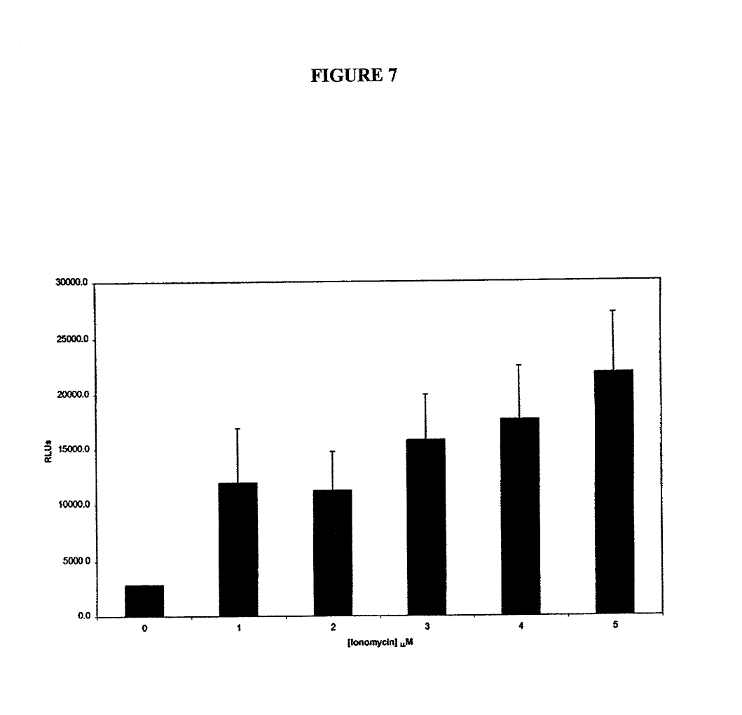

Example 3: Ionomycin is a calcium ionophore that acts as a mobile ion carrier for Ca²⁺. We investigated increasing concentrations of this agent on K562 cells. As with the previous examples, the cells were set up in 100 μl volumes in triplicate wells of a 96 well Isoplate™. The concentrations of ionomycin (Sigma) used were 1, 2, 3, 4 and 5 mM. Cells were incubated for three hours in the presence of the agent prior to determination of AK activity. The data shown in FIG. 7 are for the 1-second integral reading taken at time 0 using the Wallac MicroBeta Jet. As this figure shows, there was a concentration dependent effect of the ionophore, with the lowest concentration generating a significant increase in cytolysis above that found with the control cells not exposed to the agent. Again the control cells continue to proliferate and the results show very little AK activity associated with a healthy, actively dividing population.

If required, it would be possible to use a detergent or sonication or some other means of releasing all of the AK from the initial starting cell number. This would allow for a calculation of a percentage of cytolysis compared to controls to be made for each model. It would therefore be possible to supply a suitable releasing reagent in the kit contents (see appendix 1 for formulation).

Determination of Cytolysis by a Physical Agent

There are a number of ways of inducing primary necrosis including heat treatment (see previous examples), sonication and freeze/thaw. Examples of these data have been highlighted above.

Biocompatibility Testing

There are a wide variety of agents that can be used to provide vehicles for engraftment of cells, or act themselves as replacement material for example heart valves and hip replacement. New biocompatibility agents are constantly being investigated, but at present there is no widespread usage of a test, which will show whether these agents have the potential to cause damage to cells or initiate a cell mediated reaction. ATP bioluminescence measurements can be used to determine possible cytotoxic effects, however, this assay may not be sufficient to detect cytolysis in a small population of cells. Because of the amplification of signal associated with AK it is possible to detect a low level of cytotoxic activity. This is important if a graft is expected to last for the natural life of an individual. If there were any leaching effects of chemicals that could cause cytolysis, then this would lead to damage surrounding the engraftment.

Using the methods of the invention, it is possible to test not only chemical compounds, but also solid materials, for example, porous silicon discs. Autoclaving of these discs can result in a change in the surface of the discs and renders them toxic to lymphocytes. The AK assay could be performed in this situation where the amount of AK activity would correlate with cytolysis, this would indicate a lack of suitability of a biocompatible compound.

High Throughput Screening

This would predominately relate to the use of soluble agents to induce cytotoxicity as described above. The assay can be used in the small volumes associated with 384 and 1536 well plates, in addition to the 96 well plate format. Under these circumstances, where laboratory robots are used, the assays would be prepared in a large number of plates. The assay could then be carried out using the robots to transport the plates into a luminometer with injectors, and the assay performed as described above.

Another option arises due to the long half-life of the ‘glow’ of light from the reaction. Once the reaction has plateaued, emitted light intensity the remains substantially constant. This allows for the AKR to be added to the plates in batches, so the plates can be read even 3-4 hours after addition of the reagents.

A particular advantage of the AK method of the invention is the use of one reagent with no separate detergent/lysis steps. Detergents can cause problems with frothing in the lines and dispensing tips on the robots and sample processors used.

For this application according to the invention cells would preferably be incubated in clear bottomed white or black walled plates, or even all white/black plates with the agent of choice, at the concentration and incubation time required. With respect to the use of smaller volumes for 384 well plates (maximum volume per well 60 μl). The assay of the invention have been performed with total reaction volumes of 20 μl (10 μl of sample plus 10 μl of AK reagent), but there is no reason why total smaller reaction volumes of 6-10 μl cannot be used, which enables 1536 well microtitre plates to be employed according to the invention.

Cellular Effectors—Cell Mediated Cytotoxicity

Cell mediated cytotoxicity can be induced in a number of different ways leading to a variety of applications. For example, the generation of cytotoxic T lymphocytes (CTLs) from their precursors results in a population of cells capable of specifically recognizing and lysing target cells. CTLs have been identified that can kill bacterially or virally infected target cells. They can kill tumour cells both in vitro and in vivo, CTLs can cause cytolysis of their targets in both an MHC-mediated specific manner, or in a non-specific way. Other cell types can also be induced to kill target cells; tumour infiltrating lymphocytes are able to release a number of agents that activate macrophages for tumour cell killing. Natural Killer (NK) cells can be activated to kill different target cell lines in vitro, examples of these target cells include the most commonly used Daudi (lymphoblastoid) and K-562 cell lines (erythroleukemia).

Another system where determination of cell cytotoxicity is important is leukaemia and bone marrow transplantation. In an attempt to minimize graft versus host disease in bone marrow transplantation, and the amount of immunosuppressive therapy required after transplantation, there is a need for sensitive assays of CTL precursors. Under these circumstances the donor cells are used as responders, and the recipient cells as stimulators. Limiting dilution assays are then performed with the recipient cells used as the targets.

In all of the above cases the induction of cell membrane disruption, as a result of the actions of effector cells, will lead to the release of adenylate kinase from the target cells which can be detected according to the methods of the invention.

There is no requirement for the removal of cells from the assay reaction mixture of the invention, unlike known commercial lactate dehydrogenase assays, where cells must be spun down and only the cell free supernatant used. In order to determine maximal AK activity in the cells a sample of target cells need to be lysed with a triton X-100 containing detergent (see appendix 1).

Example 4 Incubation of murine cytotoxic lymphocytes with a number of different transgenic mice target cells. The plates were set up with cells in 200 μl volumes and effector:target (E:T) ratios of 50:1, 25:1, 12:1, 6:1, and 3:1. For determination of spontaneous release, separate wells were set up with effector or target cells alone. In order to determine the maximal release of AK from the targets, a separate set of triplicates was set up for the addition of a Triton X100 based detergent to release all of the AK. It is then possible to calculate the percentage kill by taking account of the stimulated AK release as a percentage of the maximal release above the spontaneous release of both cell populations. The plates were centrifuged at ×400 g for 10 minutes and then 100 μl of cell supernatant was harvested and assayed for AK activity as described above with the AKR. The results from this assay were compared with the more conventional ⁵¹Cr release and showed a correlation with this radiometric detection (see Table 1).

TABLE 1

| E:T Ratio | AK Assay | ⁵¹Cr |
|---|---|---|
| 50:1 | 71% | 75% |
| 25:1 | 72% | 67% |
| 12:1 | 41% | 53% |
| 6:1 | 32% | 37% |
| 3:1 | 29% | 29% |

The figures shown in Table 1 relate to the % cytotoxicity calculated from the means of triplicate samples for each experimental condition.

For the AK assay the calculation of percentage cytotoxicity was calculated as follows:

E:T activity−Effector Spontaneous−Target Spontaneous×100

(Target Max.−Target Spontaneous)

wherein

‘E:T activity’ is the release of AK as a result of mixing the two cell populations together and inducing cytolysis,

‘Effector Spontaneous’ is the spontaneous release of AK from the effector cells alone,

‘Target Spontaneous’ is the spontaneous release of AK from the target cells alone, and

‘Target Max’ is the total AK available to be released from the target cells, determined after lysis with a suitable detergent containing buffer.

For all data points, the AK activity in the complete medium was subtracted.

The assay could be performed in this way for all cell mediated cytotoxicity. The assay is particularly useful for determination of antibody-dependent cytotoxicity and complement mediated/complement-dependent cell cytotoxicity.

Antibody-induced effector mechanisms are mediated by phagocytes, NK cells or the complement system. Compared with lymphocyte recognition systems, the complement system has a more limited way of discriminating foreign invaders, through molecules that regulate complement activation on the surface of cells An example of this is the complement-dependent cytotoxicity of tumour cells after binding of Clq (complement protein) to immunoglobulins (either IgG or IgM) on the tumour cell surface, resulting in lysis of the cell membrane. The effectiveness of this form of tumour cell killing could be determined using the methods of the invention for the measurement of AK from the tumour cells exposed to activated complement components. This form of immunity mediated killing does not require the presence of effector cells, unlike complement-dependent cell cytotoxicity where phagocytic cells (monocytes, polymorphonuclear leukocytes) and NK cells, once activated by complement components, will bind to complementary receptors on tumour cells, and bring about cell lysis.

The assay methods and kits of the invention have a number of advantages, some of which are listed below:

The measurement of AK activity can be carried out in culture supernatants and in the presence of cells.

A one step bioluminescence based reagent is a great time saving convenience.

AK activity can be measured immediately after addition of reagent. Alternatively, the kinetics of the light output can be adjusted to allow for addition of reagent, and then determination of RLUs up to 5 hours later, with the addition of a suitable internal standard for AK activity.

There is no requirement for highly pure ADP or luciferase reagent, nor is there a requirement for additional magnesium ions.

The assay performs accurately at low ADP concentrations.

The assay performs well in the small volumes associated with 384 well plate technology.

The assay can be performed in the presence of a Triton x100 based detergent.

The reagent is capable of being lyophilised.

The formulation of the AKR most commonly used according to the invention is shown in appendix 1.

Lists of the suppliers cited in the text are shown in appendix 2.

### Appendix 1

ATP Monitoring Reagent (AMR) Formulation

| Reconstituted AMR | | | |
|---|---|---|---|
| Magnesium acetate | 20 | mM | Sigma |
| Tetrasodium pyrophosphate | 8 | μM | Sigma |
| Bovine Serum Albumin | 0.32% | w/v | Sigma |
| D-Luciferin | 712 | μM | ConCell |
| L-Luciferin | 17.8 | μM | ConCell |
| Luciferase | 17 | nM | Europa Bioproducts |
| Dextran | 3 | mg ml⁻¹ | Sigma |
| Tris | 40 | mM | Sigma |
| EDTA | 800 | μM | Sigma |
| Final Reaction Concentrations | | | |
| Magnesium acetate | 5 | mM | |
| Tetrasodium pyrophosphate | 2 | μM | |
| Bovine Serum Albumin | 0.08% | w/v | |
| D-Luciferin | 178 | μM | |
| L-Luciferin | 4.45 | μM | |
| Luciferase | 4.24 | nM | |
| Dextran | 750 | μg ml⁻¹ | |
| Tris | 10 | mM | |
| EDTA | 200 | μM | |

Tris Acetate (TA) Buffer (Sufficient for 1 Liter)

| | | |
|---|---|---|
| Tris | 12.1 | g |
| EDTA | 0.744 | g |

0.1M Tris, 2 mM EDTA adjust to pH 7.75 with glacial acetic acid.

Detergent Releasing Reagent (1 Liter)

| | | | |
|---|---|---|---|
| EDTA | 0.744 | g | |
| Triton x100 | 2.5 | ml | |
| Dithiotbreitol | 0.14 | g | Sigma |

Dissolve in Tris acetate buffer pH 7.75.

Adenylate Kinase Reagent

| | | |
|---|---|---|
| ADP | 100 | μM |
| Mg Ac | 2 | mM |
| AMR as above for reconstituted reagent. | | |

Ideally 100 μl of this reagent would be added to 100 μl of cells, but most of the examples were performed with 50 μl of cell samples and 150 μl of AKR.

### Appendix 2: Suppliers

Biotrace Ltd

The Science Park

Bridgend

CF31 3NA

Calbiochem-Novabiochem (UK) Ltd

Boulevard Industrial Park

Padge Road

Beeston

Nottingham

NG9 2JR

ConCell BV

Wevelinghoven 26

Nettetal

D-41334

Germany

Europa Bioproducts Ltd

Europa House

15-17 North Street, Wicken

Ely, Cambridge

CB7 5XW

Fahrenheit Lab Supplies

Northfield Road

Rotherham

South Yorkshire

S60 1RR

Labtech International Ltd

1 Acorn House

The Broyle

Ringmer

East Sussex

BN8 5NW

Labsystems Oy

Sorvaajankatu 15

Helsinki

Finland

00810

Perkin Elmer Life Sciences

Perkin Elmer House

204 Cambridge Science Park

Cambridge

CB40GZ

Sigma-Aldrich Co Ltd

Fancy Road

Poole

Dorset

BH12 4QH

Wallac Oy

PO Box 10

Turku

FI-20101

Finland

European Collection of Cell Cultures (ECACC)

Department of Cell Resources

Salisbury

UK

American Type Culture Collection (ATCC)

Rockville

Md. 20852

USA

1. An in vitro method of detecting the cytotoxic activity of an effector on a non-microbial target cell comprising providing a sample containing the target cell; treating the target cell with the effector; and detecting adenylate kinase in the sample, wherein cytotoxic activity is indicated by an increase in the amount of adenylate kinase in the sample of the effector treated target cell sample as compared with adenylate kinase in a sample of an untreated target cell.

2. The method of claim 1 wherein adenylate kinase in the target cell sample is detected by adding ADP and detecting its conversion to ATP using a bioluminescent reagent comprising luciferin or a derivative thereof and a luciferase, said luciferin or a derivative thereof emitting light in a bioluminescent reaction with the luciferase in the presence of ATP, whereby the emitted light intensity is measured to determine an ATP concentration.

3. The method of claim 2 wherein the effector is selected from the group consisting of a cellular effector and a non-cellular effecctor.

4. The method of claim 3 wherein the effector is cytotoxic T lymphocyte (CTL).

5. The method of claim 3 wherein the non-cellular effector is one or more compounds produced by and optionally secreted from an animal cell.

6. The method of claim 4 or 5 wherein adenylate kinase is detected in the presence of animal target cells.

7. The method of claim 6 wherein adenylate kinase is detected in the presence of mammalian target cells.

8. The method of claim 7 wherein the target cell is a cancer cell.

9. The method of claim 8 wherein the ADP concentration is in the range of 0.5 μM to 125 μM.

10. The method of claim 9 wherein light detection is carried out as soon as possible after addition of the bioluminescent reagent.

11. The method of claim 9 wherein light detection is carried out when the light output following addition of the bioluminescent reagent is substantially stable.

12. The method of claim 11 wherein light detection is carried out up to 5 hours after addition of the bioluminescent reagent.

13. The method of claim 3 wherein the method is employed in high throughput screening of one or more effectors.

14. The method of claim 13 wherein the sample, effector and bioluminescent reagent comprise a combined total reaction volume of from 6 μl to 300 μl.

15. The method of claim 14 wherein the combined total reaction volume is equal to or less than 70 μl.

16. The method of claim 14 wherein the combined total reaction volume is equal to or less than 20 μl.

17. The method of claim 1 wherein the target cells are subjected to one or more further tests to determine properties of the cells.

18. A kit for detecting the cytotoxic activity of an effector on an non-microbial target cell by detecting adenylate kinase comprising a multiwell microtitre plate; a sample containing an non-microbial target cell; ADP; and a bioluminescent reagent for detecting adenylate kinase by luminescence detection means.

19. The kit of claim 17 wherein the multiwell microtitre plate contains 96 wells or more.

20. The kit of claim 18 wherein the multiwell microtitre plate contains 384 or 1536 wells.

21. The kit of claim 19 wherein each well of the microtitre plate has a volume selected from the group consisting of 300 μl or less for a 96 well plate; 70 μl or less for a 384 well plate; and 10 μl or less for a 1536 well plate.

22. The kit of claim 21 wherein the volume of each well of the microtitre plate is from 6-10 μl.

23. The kit of claim 22 wherein the volume of each well of the microtitre plate is from 6-8 μl.

24. The kit of claim 19 wherein the reagent for detecting adenylate kinase comprises ADP and a bioluminescent reagent which produces light in the presence of ATP.

25. The kit of claim 24 wherein the bioluminescent reagent for detecting adenylate kinase is provided as a single combined solution.

26. The kit of claim 25 wherein the bioluminescent reagent is provided in lyophilised form.

27. The kit of claim 26 further comprising a buffer for reconstituting, diluting or dissolving the bioluminescent reagent and ADP; the buffer, ADP and bioluminescent reagent preferably being provided in proportions not exceeding those required to carry out a predetermined number of methods, as claimed in claim 2, in a total assay reaction volume not exceeding 300 μl, provided that the buffer may be present in the kit in excess of its required volume, preferably up to 80% by volume.

28. A combined reagent for detecting adenylate kinase in a sample comprising a solution of ADP 100 μM and magnesium acetate 2 mM mixed with

| | | |
|---|---|---|
| Magnesium acetate | 5 | mM |
| Tetrasodium pyrophosphate | 2 | μM |
| Bovine Serum Albumin | 0.08% | w/v |
| D-Luciferin | 178 | μM |
| L-Luciferin | 4.45 | μM |
| Luciferase | 4.24 | nM |
| Dextran | 750 | μg ml⁻¹ |
| Tris | 10 | mM |
| EDTA | 200 | μM |

29. The combined reagent of claim 27 wherein the solution is lyophilised.

30. The method of claim 2 wherein the bioluminescent reagent is the combined reagent of claim 28.
